(19) United States
(12) Patent Application Publication
(10) Pub. No.: US 2014/0065106 A1
(43) Pub. Date: Mar. 6, 2014

(54) Blood Plasma Based Hydrogels for Tissue Regeneration and Wound Healing Applications

(71) Applicant: BOARD OF REGENTS, THE UNIVERSITY OF TEXAS SYSTEM, Austin, TX (US)

(72) Inventors: Laura Suggs, Austin, TX (US); Shanmugasundaram Natesan, San Antonio, TX (US); Robert Christy, San Antonio, TX (US); David Zamora, San Antonio, TX (US)

(73) Assignee: BOARD OF REGENTS, THE UNIVERSITY OF TEXAS SYSTEM, Austin, TX (US)

(21) Appl. No.: 13/970,883

(22) Filed: Aug. 20, 2013

Related U.S. Application Data
(60) Provisional application No. 61/695,561, filed on Aug. 31, 2012.

(51) Int. Cl. A61K 35/16; A61K 9/00; A61K 38/48; A61K 35/12

(52) U.S. Cl. CPC A61K 35/16; A61K 35/35; A61K 9/0024; A61K 38/4833
USPC 424/93.7; 424/530; 424/94.64

(57) ABSTRACT

The present disclosure generally relates to tissue engineering and wound healing. More particularly, the present disclosure relates to the modification of plasma with a stability conferring agent to create a hydrogel for use in regenerative medicine and other tissue engineering applications.

### CROSS-REFERENCE TO RELATED APPLICATIONS

This application claims the benefit of U.S. Provisional Application Ser. No. 61/695,561 filed on Aug. 31, 2012, which is incorporated by reference.

### STATEMENT OF GOVERNMENT INTEREST

None

### BACKGROUND

Biomaterials are any substance (other than a drug) or combination of substances, synthetic or natural in origin, which can be used for any period of time, as a whole or as a part of a system which treats, augments, or replaces any tissue, organ, or function of the body. Biomaterials provide the underpinning of many biomedical technologies, particularly in regenerative medicine.

### SUMMARY

The present disclosure generally relates to tissue engineering and wound healing. More particularly, the present disclosure relates to the modification of blood plasma to create a hydrogel for use in regenerative medicine and other tissue engineering applications.

In one embodiment, the present disclosure provides a modified plasma comprising at least one stability conferring agent co-polymerized to fibrinogen present in the plasma.

In another embodiment, the present disclosure provides a method of forming a modified plasma hydrogel comprising obtaining plasma; adding a solution of stability conferring agent to the plasma to create stability conferring agent-plasma solution, wherein the stability conferring agent copolymerizes with fibrinogen present in the plasma; and initiating crosslinking of stability conferring agent-plasma solution to form a modified plasma hydrogel.

In another embodiment, the present disclosure provides a system comprising: a modified plasma hydrogel; and therapeutic cells in contact with the hydrogel, wherein the therapeutic cells are capable of differentiating into vascular-like structures.

In another embodiment, the present disclosure also provides a reagent kit comprising polyethylene glycol; tris-buffered saline; and a calcium solution or a thrombin solution.

The features and advantages of the present invention will be apparent to those skilled in the art. While numerous changes may be made by those skilled in the art, such changes are within the spirit of the invention.

### DRAWINGS

Some specific example embodiments of the disclosure may be understood by referring, in part, to the following description and the accompanying drawings.

FIG. 1 is a photograph showing plasma obtained through a volunteer donor.

FIG. 2 is a photograph depicting the clarity of plasma as platelet-rich plasma before centrifugation (left), and after (right). Notice the gradation lines are apparent in the platelet-free plasma tube, but not in platelet-rich plasma.

FIG. 3 is a photomicrograph demonstrating the presence of red blood cells and platelets in platelet-rich plasma (left) or absence of these elements in platelet-free plasma (right), as observed using a standard tissue culture microscope and a hemocytometer grid.

FIG. 4 shows a histogram showing a step by step quantification of red blood cells and platelets in whole blood, platelet-rich plasma, and platelet-free plasma preparations. A representative sample is depicted in the graph.

FIG. 5 is a photograph depicting the rigidity and clarity conferred to a PEGylated platelet-free plasma gel (left) versus native platelet-free plasma (right).

FIG. 6 is a photomicrograph depicting potential therapeutic cells growing and forming networks in both PEGylated platelet-rich plasma, and PEGylated platelet-free plasma over an 11 day period. Here we show an example of human adipose derived stem cells (ACSs) growing in the 3D matrices, but this technology applies to any cell type desired. Notice, the cells in the platelet-free plasma are better able to form networks than cells in the platelet-rich plasma matrix.

FIG. 7 is a graph showing storage modulus of the PEGylated PFP gels prepared by gelation with different concentrations of (A) CaCl₂ and (B) thrombin.

FIG. 8 shows light microscopic images of differentiation time-course of ASCs into vascular like structures in PEGylated plasma hydrogels prepared with different concentration of thrombin.

FIG. 9 shows light microscopic images of differentiation time-course of ASCs into vascular like structures in PEGylated plasma hydrogels prepared with different concentration of CaCl₂.

FIG. 10 shows light photomicrograph images of the stem cell isolation process from adipose tissue obtained from a normal individual or a burn patient. ASCs in PEGylated PFP plasma hydrogels.

FIG. 11 scanning electron microscopy (SEM) images of the morphological composition of fibrin and PEGylated plasma hydrogels.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure generally relates to tissue engineering and wound healing. More particularly, the present disclosure relates to the modification of plasma to create a hydrogel for use in regenerative medicine and other tissue engineering applications.

Blood plasma or plasma is the yellow or gray-yellow, protein-containing fluid portion of blood in which the blood cells and platelets are normally suspended. Plasma contains fibrinogen, or its derivative, fibrin. Fibrin is the biopolymer formed after thrombin-mediated cleavage of fibrinogen and is naturally present in solution within plasma. Upon activation of platelets, fibrin is cleaved from its parent fibrinogen molecule to congeal into a type of gel glue that seals breached vascular structures, and helps to naturally form the protective dermal scab of an open wound. The present disclosure is based, in part, on this basic property of plasma, which has been underappreciated in the field of regenerative medicine.

The fibrin biopolymer itself, however, suffers from low mechanical stiffness, contraction, and rapid degradation; which do not allow the proper formation of tissue engineered structures. To overcome these problems, according to the present disclosure, the fibrinogen in plasma may be modified before use to serve as a better three dimensional tissue engineering scaffold. One such approach modifies fibrinogen by copolymerizing it with polyethylene glycol (PEG). Such PEGylated fibrins exhibit unique features of both synthetic hydrogels and natural materials. Specifically, (1) the presence of PEG provides a highly hydrated (>90% water) moist environment for managing exudates, (2) the presence of fibrin confers biodegradability to the material; however, our results have shown that it is significantly more stable in vitro than fibrin alone, and (3) the inherent biologic activity of fibrin encourages the natural healing process in hosts by stimulating tissue and blood vessel in-growth. This matrix system is therefore able to be responsive to cell-mediated remodeling while allowing for handling and storage under a variety of conditions.

The present disclosure provides, according to certain embodiments, compositions comprising PEGylated plasma or plasma in which at least a portion of the fibrinogen present in the plasma is co-polymerized with polyethylene glycol. Such PEGylation of the blood plasma (copolymerizing the fibrinogen with polyethylene glycol) allows for the formation of plasma hydrogels. The PEGylation of the blood plasma serves as a secondary crosslinking mechanism to form robust elastic hydrogels upon crosslinking with a fibrinogen converting agent (e.g., thrombin or through the addition of calcium). Such compositions may be useful for, among other things, wound repair and healing, drug delivery, and tissue engineering.

Any plasma containing fibrinogen may be used according to the present disclosure. In certain embodiments, commercially available plasma may be used. In other embodiments, the plasma may be obtained from an allogenic source such as, for example, a blood bank. In other embodiments, the plasma may be obtained from an autologous source such as, for example, a donor. In another embodiment, plasma is obtained from umbilical cord-blood. The plasma may fresh, i.e., used shortly after collection. For example, the plasma may be fresh frozen plasma. Alternatively, the plasma may stored prior to use (e.g., frozen plasma).

Generally, the plasma will include an anticoagulant. Normally, thrombin is present in plasma but is inactivated through the use of an anticoagulant agent administered during collection of the blood so as to prevent blood coagulation. Accordingly, in certain embodiments, the plasma may contain an anticoagulant such as, for example, heparin, citrate phosphate dextrose adenine (CPDA), acid-citrate-dextrose (ACD), and citrate phosphate dextrose (CPD) solutions.

In certain other embodiments, the plasma used in conjunction with the present disclosure may be platelet-rich plasma. In certain embodiments, it may be desirable to use platelet free plasma (PFP) rather than platelet rich plasma (PRP). Platelet rich plasma may be centrifuged in order to collect supernatant. The supernatant is the platelet free plasma. A point-of-care device such as, for example, Arteriocyte Medical Systems Magellan® Autologous Platelet Separator, may be used to separate platelet rich plasma. In certain embodiments, the plasma supernatant may be inspected for purity. Any method known in the art may be used to confirm purity of the supernatant (platelet free plasma). Such methods may include using a hemocytometer and a microscope or a hematology analyzer.

PEG is a nontoxic and amphiphilic compound, i.e. soluble both in water and in most organic solvents. Protein PEGylation is generally achieved via stable covalent bonds between an amino or sulfhydryl group on a protein and a chemically reactive group (carbonate, ester, aldehyde, or tresylate) on the PEG. The resulting structures can be linear or branched. The reaction can be controlled via factors such as protein type and concentration, reaction time, temperature, and pH value. Environmental factors such as these likewise influence electrostatic binding properties and protein charge, form, and size.

The PEG is added to the plasma to enable a stable hydrogel to form. While PEG is the preferred molecule for copolymerization with fibrinogen, other reactive derivatives of a water soluble polymer, such as, for example, polyvinyl alcohol, polyhydroxyethyl methacrylate, hyaluronic acid, or alginate also may be suitable.

The PEG may be any suitable PEG capable of copolymerizing with fibrinogen. Examples of suitable PEGs include, but are not limited to, difunctional N-hydroxysuccinimide (NHS)-PEG, difunctional benzoyltriazole carbonate (BTC)-PEG, difunctional succinimidyl carbonate (SC)-PEG, and difunctional succinimidyl methyl butanoate (SMB)-PEG, succinimidyl succinate (SS)-PEG; and succinimidyl glutarate (SG)-PEG. Suitable PEGs also may be bifunctional.

The PEG may be added to obtain a final PEG concentration of from about 400 μg/mL to about 2000 μg/mL of plasma. In certain embodiments, PEG may be added to the plasma to obtain a final PEG concentration of about 800 μg/mL of plasma. One of ordinary skill in the art with the benefit of this disclosure, will be able to recognize the appropriate concentration of stability conferring agent suitable for specific applications.

In certain embodiments, prior to hydrogel formation, other components may be added to the PEGylated plasma. Such biologics may include, but are not limited to, growth factors, extracellular matrix proteins, therapeutic drugs, and antibiotics.

In other embodiments, prior to hydrogel formation, therapeutic cells may be added to the PEGylated plasma. In certain other embodiments, therapeutic cells may be added after hydrogel formation. The therapeutic cells may be obtained from an autologous source. In certain embodiments, the therapeutic cells may be stem cells. In certain embodiments, the therapeutic cells may be bone marrow derived stem cells, adipose derived stem cells, induced pluripotent stem cells, foreskin fibroblasts, endothelial cells, stromal vascular fraction (SVF), or combinations thereof. In certain embodiments, the cells may be adipose derived stem cells from debrided burn skin. In certain embodiments, a combination of cells may be added. One of ordinary skill in the art, with the benefit of this disclosure, will be able to recognize suitable combinations of cells that may be used in conjunction with the present disclosure. The cells may be added at a concentration of from about 25,000 cells/mL of gel to about 5,000,000 cells/mL of gel. One of ordinary skill in the art with the benefit of this disclosure, will be able to recognize the appropriate concentration of cells suitable for specific applications. The therapeutic cells may grow proliferate or extend cellular processes on or inside the PEGylated plasma hydrogels of the present disclosure and form networks.

In certain embodiments, the present disclosure provides compositions comprising PEGylated plasma and fibrinogen-converting agent. By combining the PEGylated plasma solution with a solution containing a fibrinogen-converting agent a PEGylated plasma hydrogel may be formed. Without being bound by a particular mechanism, fibrinogen in the fibrinogen solution is converted to fibrin through a proteolytic reaction catalyzed by a serine protease in the serine protease solution. Fibrin monomers then aggregate to form a PEGylated plasma hydrogel. To overcome the effects of the anti-coagulant, and trigger formation of the PEGylated plasma hydrogel, fibrinogen converting agents may be added to the system (e.g., exogenous calcium or thrombin). Fibrinogen converting agents include without limitation, proteases such as serine proteases (e.g., thrombin), CaCl₂, or combinations thereof. Other fibrinogen-converting agents suitable for converting fibrinogen to fibrin include, without limitation, mutant forms of thrombin exhibiting increased or decreased enzymatic activity. In certain embodiments, the calcium added may be in the form of CaCl₂. In certain embodiments, the CaCl₂ may be added to achieve a final concentration of from about 5 mM to about 40 mM, from about 15 mM to about 30 mM, and from about 11 mM to about 27 mM in the PEGylated plasma system. The CaCl₂ is capable of gelling the modified plasma mixture in approximately 20-30 minutes. Thrombin and other serine proteases may also be used to induce hydrogel formation. In certain embodiments, the thrombin may be added to the modified plasma at a concentration of from about 2 U/mL to about 25 U/mL and from about 5 U/mL to about 17.5 U/mL. The addition of thrombin at these concentrations allows for hydrogel formation of the modified plasma within about 15 minutes.

The hydrogel's flexibility can be altered by adding fibrinolytic inhibitors (e.g., tranexamic acid at 9.2% w/v, or aprotinin at 3000 KIU/ml, where KIU is kallikrein IU) or anticoagulants (e.g., trisodium citrate at 3-10 mg/ml, or glycine at 10-40 mg/ml) to either or both the solutions. In addition, such components can be used to alter the polymerization time associated with hydrogel formation.

The PEGylated plasma hydrogels of the present disclosure provides certain advantages. Their physical properties are improved over plasma that is crosslinked that does not contain PEG. These non-PEG hydrogels are weak and degrade quickly; they are also not suitable for applications such as a hemostatic agent, surgical sealant, cell, or drug delivery vehicles.

In certain embodiments, the PEGylated plasma hydrogels of the present disclosure may be used to treat an animal. In certain embodiments, the PEGylated plasma hydrogels may be used to treat a human. The use of the PEGylated plasma hydrogels of the present disclosure allows for in situ hydrogel formation and for the hydrogel to conform to the size of a wound or the size and shape of the location to be treated. The hydrogel may serve as a scaffold to promote wound healing and growth of any therapeutic cells that may be present in the system of the present disclosure. In certain embodiments, the hydrogels of the present disclosure may be used to promote organ healing or to reconstruct, either temporarily or permanently, a tissue or organ. In other embodiments, the PEGylated plasma hydrogels may be used, for example, as wound healing dressings, dermal fillers, and anti-adhesion barriers, hemostatic agents, surgical sealants, and cell or drug delivery vehicles.

The present disclosure also provides, according to certain embodiments, methods for forming PEGylated plasma hydrogels. In one embodiment, a method comprises providing a PEGylated plasma and initiating crosslinking of the PEGylated plasma to form a hydrogel.

The present disclosure also provides, according to certain embodiments, methods for using PEGylated plasma hydrogels. In one embodiment, the present disclosure provides a method comprising introducing a fibrinogen-converting agent to a PEGylated plasma and allowing the PEGylated plasma to form a hydrogel. The hydrogel may be formed in vivo or ex vivo. Such methods may be used to treat a patient.

The hydrogel, or the fibrinogen-converting agent and the PEGylated plasma, may be provided by any means of delivery. For example, delivery may be effected via spray, injection, endoscopic injection, pouring, and the like.

The present disclosure also provides, according to certain embodiments, a kit comprising PEGylated plasma and fibrinogen-converting agent. The PEGylated plasma and fibrinogen-converting agent may be packaged separately or together. For example, the PEGylated plasma and fibrinogen-converting agent may be provided in different syringes.

The present disclosure also provides, according to certain embodiments, a system comprising PEGylated plasma disposed in a first container and fibrinogen-converting agent disposed in a second container, wherein the first and second container are operably connected to allow mixing. For example, the containers may be a dual barrel syringe that allows for mixing of the PEGylated plasma and fibrinogen-converting agent upon dispensing. Any container or delivery system for mixing and ejecting a multi-component fluid mixture is suitable.

To facilitate a better understanding of the present invention, the following examples of certain aspects of some embodiments are given. In no way should the following examples be read to limit, or define, the entire scope of the invention.

### EXAMPLES

### Example 1

Materials and Methods

Platelet Free Plasma Isolation and Characterization.

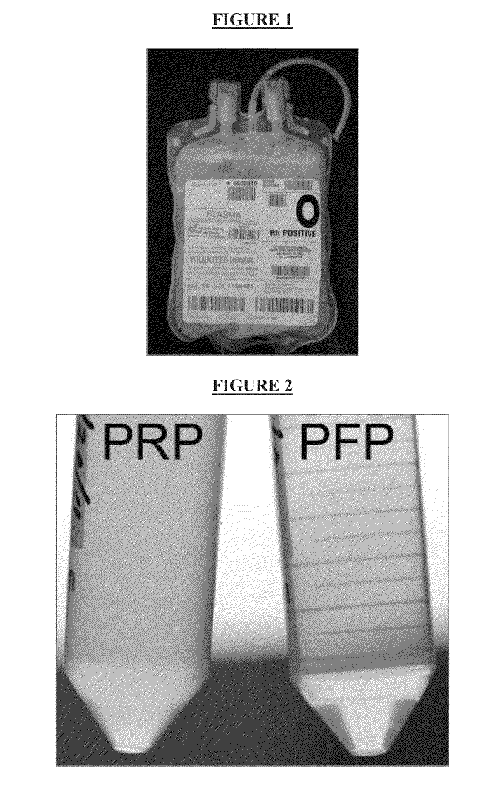

Plasma or platelet-rich plasma (PRP) was obtained from either a commercial source or from local blood bank. FIG. 1 shows plasma obtained from a volunteer donor. Frozen plasma or platelet rich plasma may be used; if frozen, the plasma or platelet rich plasma should be allowed to thaw at 37° C. for 1 hour. Once thawed, plasma or platelet rich plasma was removed from the bags and 40 ml of plasma each was placed into 50 ml conical tubes until the entire volume of plasma was placed into the tubes. The plasma was spun at 4,300×g (˜5000 rpm) for 30 min at room temperature. FIG. 2 shows the clarity of plasma as PRP before centrifugation and as PFP after centrifugation. The supernatant was collected, which is the platelet free plasma (PFP).

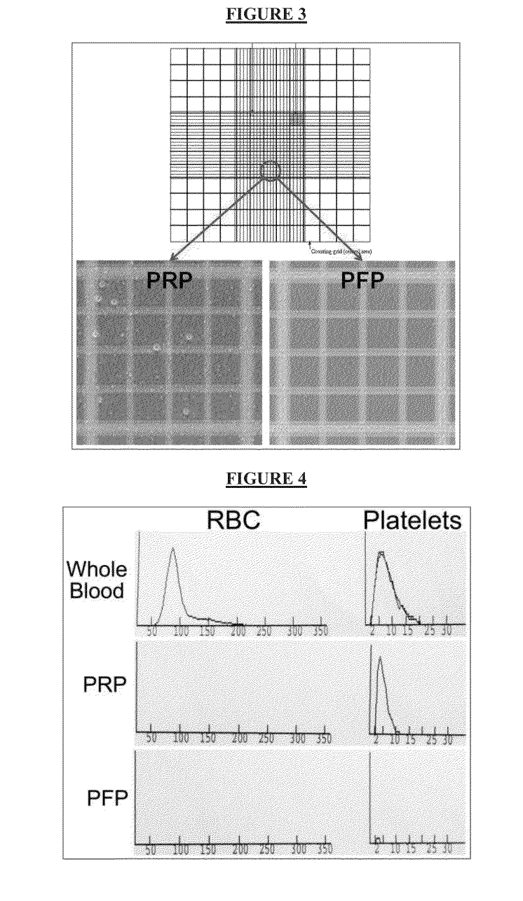

The platelet free plasma was then inspected for purity. Two methods were used to confirm the purity of the platelet free plasma: a hemocytometer and microscope (FIG. 3) and an Advia 120 hematology analyzer (Siemens) (FIG. 4) was used to determine platelet and red blood cell contamination.

Table 1 shows a summary of the donor samples obtained and used in the research and development of this technology. Samples were obtained from a commercial source South Texas Blood & Tissue Center (STBTC) or the United States Army Institute of Surgical Research (USAISR) Hematology Laboratory under IRB protocol: H-10-023. Blood type, gender, and age were provided by each source. Platelet-rich plasma (PRP) was also provided by each source and was processed by our research team as mentioned above. Hematology analysis (red blood cell & platelet counts) was also performed by the Hematology Laboratory, while Fibrinogen concentration was performed by the USAISR's Division of Laboratory Support using a Siemens Multifibren U Automated coagulation analyzer, with standard guidelines for clinical use set by the Food & Drug Administration.

TABLE 1
Human Donors for Platelet Free Plasma (PFP)

| | | | | Blood | Plasma | Platelet | Fibrinogen |
|---|---|---|---|---|---|---|---|
| Source | Accession ID | Gender | Age | Type | Type | ×10³/μl | mg/dl |
| STBTC | W140912111671 | Male | 43 | O⁺ | PFP | 3 | 277.9 |
| STBTC | W140912103684 | Male | 32 | O⁺ | PFP | 19 | 230.0 |
| STBTC | W140912111342 | Male | 20 | O⁺ | PFP | 38 | 252.2 |
| STBTC | W140912102961 | Female | 27 | O⁻ | PFP | TBD | TBD |
| STBTC | W140912105721 | Female | 38 | O⁺ | PFP | TBD | TBD |
| STBTC | W140912105723 | Female | 48 | O⁺ | PFP | TBD | TBD |
| USAISR | 911002184-D5 | Male | 33 | A⁺ | PFP | TBD | 226.0 |
| USAISR | 911002235-D6 | Male | 27 | A⁺ | PFP | 1 | TBD |
| USAISR | 911002363-D7 | Male | 37 | O⁺ | PFP | 3 | 230.0 |
| | Averages: | | 33.88 | | | 12.8 | 243.22 |

Preparation & Characterization of PEGylated PFP Hydrogels.

Polyethylene glycol (PEG) stock solution was prepared as previously published (S. Natesan, G. Zhang, D. G. Baer, T. J. Walters, R. J. Christy, and L. J. Suggs. Tissue Engineering Part A. April 2011, 17(7-8): 941-953) by dissolving the succinimidyl glutarate modified polyethylene glycol (PEG; 3400 Da) using 8 mg/mL of tris-buffered saline (TBS, pH 7.8) and filter sterilized with a 0.22-μm filter just before starting the experiment. Dissolved PEG is only effective in this application for the first 3-4 hours.

900 μl of PFP or PRP and 100 μl of PEG stock were mixed in a culture well of a 6-well plate and incubated for 10 minutes in a 5% CO₂ humidified incubator at 37° C.

Optional Addition of Therapeutic Cells.

Prior to hydrogel formation, a stock of cell suspension of desired cell density in no more than a 15-100 μl volume may be prepared; add therapeutic cells (i.e. stem cells, etc) to the PEG-PFP or PEG-PRP solution. Final concentration of cells should be approximately 25,000 to 100,000/ml of gel.

Gelation Using Calcium Chloride.

1M Calcium Chloride solution was prepared. The CaCl₂ solution was added to the PEG-PFP/cell solution or PEG-PRP/cell solution so as to have a final CaCl₂ concentration of 11 mM to 27 mM per ml of gel solution mixture. The solution was triturated once so as to ensure even mixture of the solutions and placed into a 5% CO₂ humidified incubator at 37° C. and allowed to gel for about 20-30 minutes.

Gelation Using Thrombin.

A thrombin stock solution of 100 U/mL was prepared. Add the thrombin solution to the PEG-PFP/cell solution so as to have a final concentration of 5-12.5 U/PEG-PFP/PRP solution mixture. Triturate the solution once so as to ensure even mixture of the solutions and place it into a 5% CO₂ humidified incubator at 37° C. and allow it to gel for about 15 minutes.

Since the gelation times can be fast for both processes, it is important to not hold the gel solution within the pipette tip for more than 5 seconds. Regardless of which type of gelation process is performed, wash the PEG-PFP or PEG-PRP gels twice with a saline/buffer (like HBSS, PBS) solution to remove residual cells or unbound PEG. The gels are then ready for in vitro or in vivo application.

Results

PEGylation.

All plasma based gels (PRP, Platelet Poor Plasma (PPP), and PFP) were investigated for their ability to become PEGylated and congeal. PPP is similar to platelet free plasma, but has some quantity of platelets still remaining within the plasma. PEGylation of these plasma derivatives confers better viscoelasticity and clarity than unPEGylated plasma hydrogels. A concentration dependent gelation was observed (from 400 μg/ml to 2000 μg/ml, with final PEG concentration of 800 μg/ml was found to be optimal to obtain stable gels. PEGylation of these plasma products can be accomplished within 5-10 minutes. Higher concentrations of PEG (>800 μg/1 ml of PFP or PRP) results in loss of gel viscosity.

Therapeutic Cells.

We have tried with success a multitude of human cells in or on these plasma preparations: bone marrow derived stem cells, adipose derived stem cells, foreskin fibroblasts, endothelial cells. Human dsASCs (adipose derived stem cells from debrided burn skin) grew well in PFP, but not as well as in PRP. (FIG. 6.) PEG does not appear to have any bearing on this observation. The platelets influence cell network formation in the 3D PRP scaffold, but not in 3D PFP scaffolds. Human Bone Marrow Derived Stem Cells (hBMSCs) form networks relatively slower in all plasma derived scaffolds, when compared to human ASCs. Human foreskin fibroblasts (HFFs) and ASC form networks similarly within gels, regardless if culture medium contains serum or not. It appears that plasma based gels provide sufficient growth factors for their survival and ability to thrive.

PFP, regardless if used fresh or frozen and thawed, appeared to sustain cells equally. Cells grown in basal media (in this case MesenPro), without any additional supplements, sustained cells.

PFP solution is capable of self-congealing into a gel within a span of 24-48 hrs by the simple addition of cell culture medium. Gelling time decreased with increasing concentrations of plasma in the media (range 10% to 1% plasma supplementation).

Calcium Chloride.

The simple addition of CaCl₂ can gel PEGylated plasma mixture (˜20-30 min) without the addition of exogenous thrombin. Current clinical PRP literature uses 23 mM for gelation. In our current investigation of preparing hydrogels from plasma, we used concentrations of 27 mM and as high as about 40 mM, and also formed gels with concentrations as low as 11 mM CaCl₂. At CaCl₂ of less than 11 mM concentration, the gels formed were less visco-elastic and lost their aqueous content upon removal of the gels from the culture plates and this concentration was deemed to be the lowest “usable” concentration that allowed a useful gel to be formed.

Thrombin.

The exogenous addition of thrombin between 5-25 U/ml provides good gelation within 1-15 minutes. However, 12.5 U/ml of thrombin or higher allows gels to contract and remodel over a 15 day period if cells are incorporated into the gel, as determined by in vitro analysis. Concentration of 5-10 U/ml allows gels to keep original shape under similar conditions. Hydrogels prepared with thrombin of less than 5 U/mL were fragile and lost its aqueous content when removed from the culture wells. Hydrogels prepared with thrombin above 15 U quickly gelled, and proved difficult to control even gelation. Hydrogels prepared with thrombin above 15 U quickly gelled, and proved difficult to control even gelation.

Network Formation of Cells within Gels Formed Using Calcium or Thrombin.

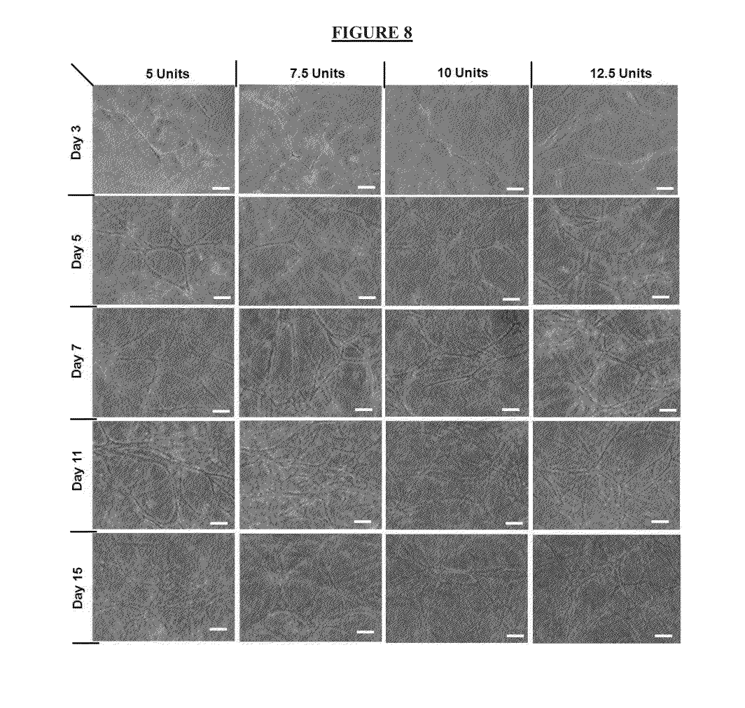

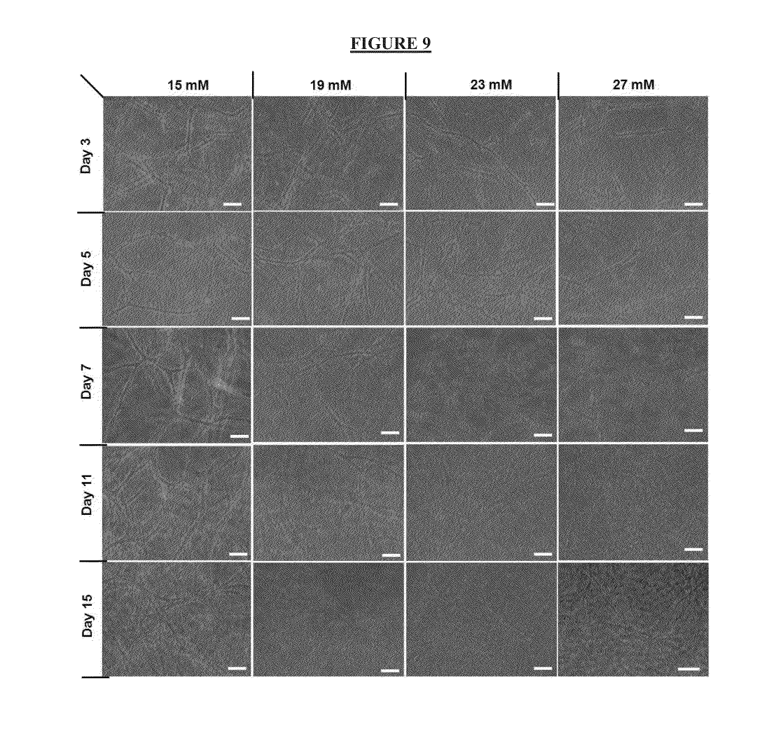

Cells within the PEGylated plasma hydrogels, gelled with either with calcium or thrombin, began to form vascular tube-like networks in the absence of additional soluble cytokines (FIGS. 8 & 9). The amount of network formation was related to the initial cell number density (50000 cells/ml in FIGS. 8 and 9); cells were able to form tubular network over time at different concentration of both CaCl₂ and thrombin. Morphologically, the networks within the thrombin based gels were more robust and thicker in diameter than those formed with CaCl₂. With different concentrations of thrombin, cells were able to sprout faster within the hydrogels made with lower concentrations of thrombin (5 U) and sustained the network formation till the time of observation (day 15). During day 15 ells were present in all the gels with different concentrations. Within the different concentrations of CaCl₂ ASCs showed morphologically thicker networks in gel made from lower concentrations of CaCl₂ (15 mM) and there was a visible change in diametric change in the vascular network formed with progressively higher concentrations of PEGylated plasma hydrogels.

Properties of PEGylated Plasma Hydrogels.

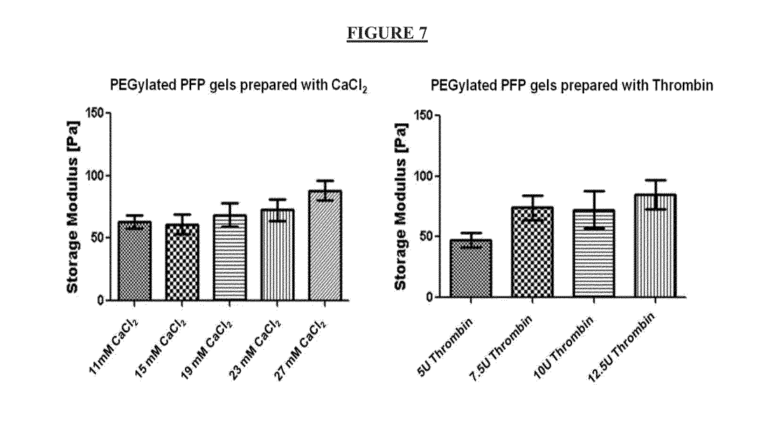

Rheological studies were carried out with PFP gels prepared using different concentrations of thrombin 5, 7.5, 10 and 12.5 U/ml concentrations. PFP gels with thrombin maintained better shape after removal from the mold that it was cast within. The storage modulus of the thrombin based hydrogels proportionally increased as initial thrombin concentrations increased, and spanned between 47 Pa to about 92 Pa. (FIG. 7). The highest storage modulus (92 Pa) was observed with 12.5 U of thrombin concentration. Above this concentration, though the gels were more stable with respect to handling and viscoelasticity, however the ASCs as they grew in vitro caused significant gel shrinkage over time.

In general, hydrogels made with CaCl₂ were more stable in terms of water retention ability and the gels with different concentrations of calcium concentration (11 mM to 27 mM) exhibited a close range in storage modulus spanning between 62 Pa to 87 Pa, with an incremental increase in storage modulus CaCl₂ concentrations increased. Though the gels prepared with higher CaCl₂ concentration were more stable with respect to complex viscosity and loss of water within the gels (FIG. 7), the ASCs within the hydrogel made with higher concentration of CaCl₂ showed networks with smaller diameters at 23 mM and above. Collectively PEGylated hydrogels made with CaCl₂ concentrations of less than 19 mM CaCl₂ exhibited consistent storage modulus.

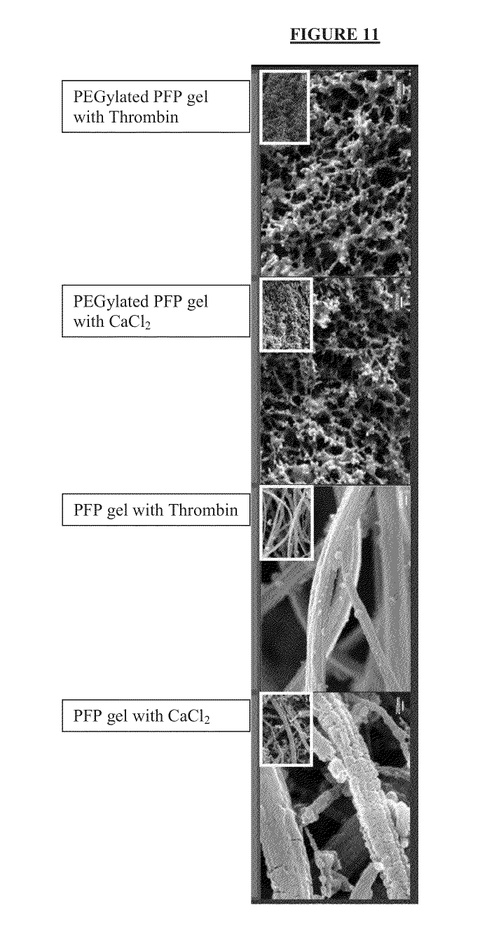

PEGylated PFP plasma hydrogels compared to PEGylated PFP gels under SEM is shown in FIG. 11. The gels were formed using 12.5 U/ml thrombin and 23 mM CaCl₂.

### Example 2

Platelet-rich plasma (PRP) and platelet-free plasma (PFP) provide patients with an autologous matrix scaffold source, and are currently being used in the treatment of articular resurfacing, tendon repair, wound healing and tissue engineering applications. We have developed novel modifications of PRP and PFP, using polyethylene glycol (PEG), that allows plasma to maintain hydrogel-like characteristics rather than an amorphous fibrin clot. Fresh PRP was provided by the Division of Hematology located at the USAISR (IRB#: H-10-023). To obtain PFP, PRP was centrifuged at 4,500×g for 30 minutes at 24° C. Both PRP or PFP were then mixed with different molar ratio of SC-PEG at a 10:1 molar ratio of PRP/PFP (based on fibrinogen concentration) to PEG for 10 minutes at 37° C. PEG-PRP/PFP was then polymerized either by adding CaCl₂ (1 mM to 30 mM) or bovine thrombin (5 U to 20 U) and their physical properties characterized. Adipose derived stem cells (ASCs) were added to PEG-PRP/PFP prior to polymerization and maintained in culture for up to 15 days.

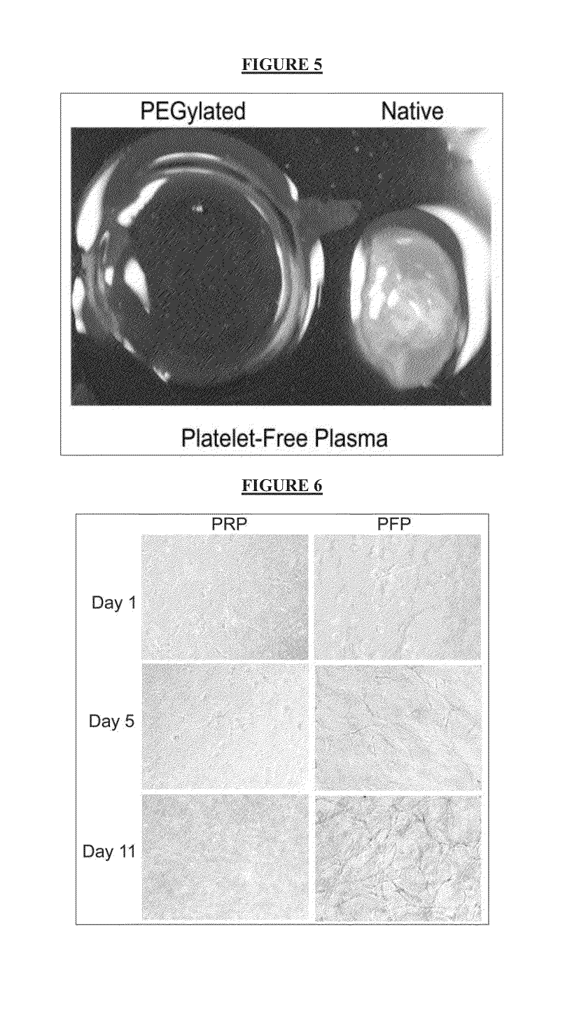

Results indicate that polymerization of PEG-PRP/PFP yielded viscoelastic, semi-rigid, clear hydrogels, while unPEGylated gels were opaque and easily deformed upon handling. (FIG. 5). Optimal concentrations for gel polymerization, which supported ASC growth over a 14 day period without the gels structural integrity becoming distorted, was within the range of 2 U to 25 U/ml of thrombin or 5 mM to 40 mM of CaCl₂, and more specifically 10 U/mL of thrombin or 23 mM of CaCl₂. At these concentrations thrombin exhibited a storage modulus of about 47 Pa to about 92 Pa, and CaCl₂ gels exhibited a storage modulus of about 62 Pa to about 87 Pa. We have demonstrated that human plasma can be PEGylated to generate a stable, viscoelastic, easy to handle and reproducible hydrogels that supports cell growth. This will allow the development of treatments using autologous patient plasma and ASCs to create a construct that can be used to treat skin wounds, regenerate skin and other soft tissue injuries

### Example 3

From a clinical stand-point, successful reconstruction of extensive skin loss requires a stable scaffolding architecture that can provide mechanical support as well as micro-environmental cues to promote granulation, vascularization, re-epithelialization and remodeling. Succinimidyl glutarate polyethylene glycol (PEG) based fibrin hydrogel induces tubular network formation of adipose derived stem cells (ASCs) within the hydrogels. Recently, we found these hydrogels with ASCs to be ‘vasculo-inductive,’ enhancing blood vessel formation during the healing process. Fibrin hydrogels with ASCs isolated from the debrided skin tissue when applied to the excision wounds increased the amount of blood vessels in the healing wound bed, compared to saline treatments and fibrin hydrogel alone. Furthermore, the blood vessels within the wound beds treated with FPEG with ASCs appeared to be larger and stained darker for von Willebrand Factor than FPEG treatments alone, suggesting that the presence of ASCs may enhance angiogenesis.

Behavior of ASCs within these PEGylated Plasma Hydrogels.

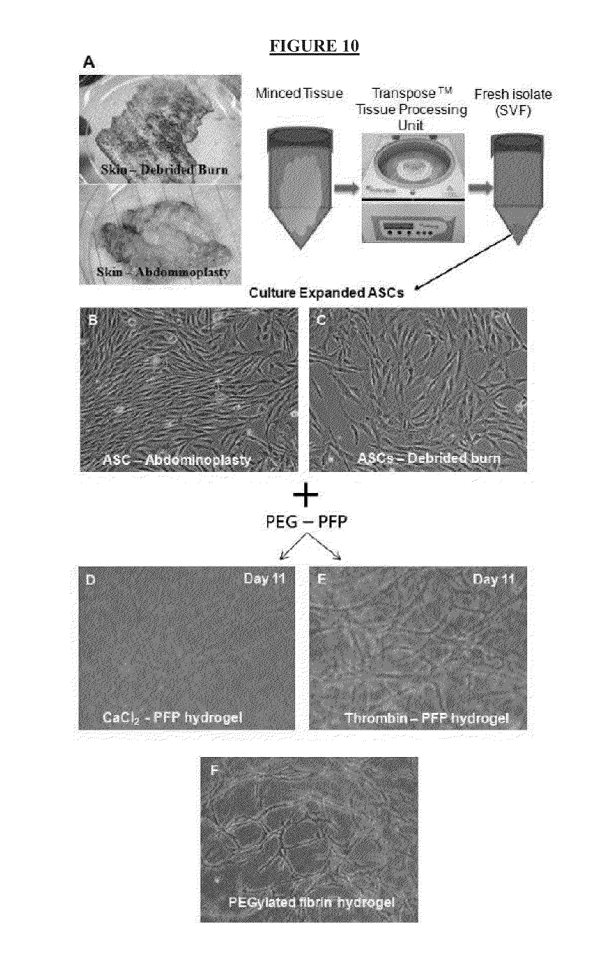

ASCs isolated from subcutaneous adipose tissue of debrided skin using a point-of-care cell isolation device were capable of forming vascular-like structures within a PEGylated fibrin matrix. In PEGylated plasma hydrogels (FIG. 10A), ASCs from abdominoplasty (FIG. 10B) and debrided skin (FIG. 10C) in a PEGylated plasma hydrogel were able to form tubular networks. FIGS. 10D and 10E are images of ASCs forming tubular networks within the plasma hydrogels prepared using CaCl₂ (23 mM) and thrombin (12.5 U), respectively. The tubular networks formed were comparable morphologically to the networks observed in the PEGylated fibrin gel (FIG. 10F).

To prepare PEGylated plasma hydrogels, succinimidyl glutarate polyethylene glycol (PEG; 3400 Da) was dissolved in tris-buffered saline (4 mg/ml, pH 7.8) and filter sterilized with a 0.22-μm filter just before starting the experiment. Plasma containing 20-25 mg of fibrinogen (observed from biochemical analysis) was mixed with PEG stock to obtain various w/w ratio mixtures (1:8, 1:10, 1:12 w/w). The mixture was then incubated for 10 minutes in a 5% CO₂ humidified incubator at 37° C. Gelation of the PEG-plasma liquid mixture was then initiated either using CaCl₂ or thrombin. To gel using CaCl₂, a 1M Calcium Chloride solution was prepared and added to the PEG-plasma solution so as to have a final CaCl₂ concentration of 15-23 mM CaCl₂/ml of gel. The mixture was then incubated for 20-30 minutes in a 5% CO₂ humidified incubator at 37° C. to obtain PEG-plasma hydrogels. To gel using the addition of thrombin, a human thrombin stock solution of 100 U/ml was added to the PEG-plasma solution so as to have a final concentration of 5 U-25 U of thrombin/PEG-plasma solution. The solution was mixed and placed into a 5% CO₂ humidified incubator at 37° C. for 15 minutes to gel.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

What is claimed is:

1. A composition comprising plasma in which at least a portion of the fibrinogen present in the plasma is co-polymerized with polyethylene glycol.

2. The composition of claim 1 wherein the plasma is from an allogenic source.

3. The composition of claim 1 wherein the plasma is platelet free plasma.

4. The composition of claim 1 wherein the plasma is platelet rich plasma.

5. The composition of claim 1 further comprising one or more components chosen from growth factors, extracellular matrix proteins, therapeutic drugs, and antibiotics.

6. The composition of claim 1 further comprising therapeutic cells.

7. The composition of claim 1 further comprising adipose derived stem cells.

8. The composition of claim 1 further comprising a fibrinogen-converting agent.

9. The composition of claim 1 further comprising a fibrinolytic inhibitor.

10. The composition of claim 1 wherein the composition is a hydrogel.

11. The composition of claim 1 wherein the polyethylene glycol is bifunctional.

12. The composition of claim 1 wherein the polyethylene glycol is SG-PEG-SG.

13. A method comprising providing a PEGylated plasma and initiating crosslinking of the PEGylated plasma to form a hydrogel.

14. The method of claim 13, wherein the PEGylated plasma is formed by copolymerizing polyethylene glycol to at least a portion of fibrinogen present in a plasma.

15. The method of claim 13, wherein the initiating crosslinking of the PEGylated plasma comprises introducing a fibrinogen-converting agent to the PEGylated plasma.

16. The method of claim 13 wherein the PEGylated plasma is formed from platelet free plasma.

17. The method of claim 13 wherein the PEGylated plasma is formed from platelet rich plasma.

18. The method of claim 13 wherein the plasma is from an allogenic source.

19. A method comprising introducing a PEGylated plasma hydrogel to a patient in need thereof.

20. The method of claim of claim 19, wherein the PEGylated plasma hydrogel forms at the site of implantation.

21. A kit comprising PEGylated plasma and fibrinogen-converting agent.

22. A system comprising PEGylated plasma disposed in a first container and fibrinogen-converting agent disposed in a second container, wherein the first and second container are operably connected to allow mixing.

23. A system comprising: a PEGylated plasma hydrogel; and therapeutic cells in contact with the PEGylated plasma hydrogel, wherein the therapeutic cells are capable of differentiating into vascular-like structures.
